(19) United States
(12) Patent Application Publication
(10) Pub. No.: US 2008/0057099 A1
(43) Pub. Date: Mar. 6, 2008

(54) Implant coatings having improved haemocompatibility

(75) Inventors: Leif Hermansson, Molle (SE); Tobias Persson, Uppsala (SE); Hakan Engqvist, Knivsta (SE)

Correspondence Address: FRISHAUF, HOLTZ, GOODMAN & CHICK, PC, 220 Fifth Avenue, 16TH Floor, NEW YORK, NY, 10001-7708, US

(73) Assignee: Doxa AB, Uppsala (SE)

(21) Appl. No.: 11/899,375

(22) Filed: Sep. 5, 2007

Related U.S. Application Data
(60) Provisional application No. 60/842,683, filed on Sep. 5, 2006.

(51) Int. Cl. A61F 2/02; A61F 2/06; A61F 2/24; A61F 2/82

(52) U.S. Cl. 424/423; 427/2.24; 427/2.25; 623/1.43; 623/2.1

(57) ABSTRACT

Implant coatings having improved haemocompatibility involve the use of alkaline chemically bonded ceramic systems based on Ca-aluminate as a thrombogenecity reducing coating material, especially intended for coating of devices used in the human or animal blood system.

### CROSS-REFERENCE TO RELATED APPLICATION

This application claims priority under 35 USC 119(e) of U.S. Provisional Patent Application Ser. No. 60/842,683 filed Sep. 5, 2006, the entire disclosure of which is incorporated herein by reference.

### TECHNICAL FIELD

For medical devices to be used in the blood system, the biocompatibility profile, with special reference to haemocompatibility, is of specific importance. Normally stents and coated stents to be used for implantation require complementary drugs in order for blood clotting behavior to be avoided. The present invention is directed to the use of Ca aluminate compositions for blood tissue implants, such as stents, which coating exhibits an improved stability in blood contact, with reduced amount of complementary drug or with no use of any complementary drugs or medication. The composition of the invention is especially intended for haemocompatible applications, and also for soft tissue applications, since both a direct blood contact and a soft tissue interaction is at hand. The invention also relates to a method of preparing the Ca aluminate coating on a blood tissue implant, coated implants obtainable by means of the method, and a method of implanting such coated blood tissue implant.

### STATE OF THE ART AND PROBLEM

Implants that are to interact with the human body should advantageously be composed of materials having a good biocompatibility. An example of such a material is the class known as “chemically bonded ceramics” (CBCs). The CBC materials are found as phosphates, aluminates, and silicates, all of which with calcium as the main cation.

Examples of such materials are the high strength CBC materials, which are based on Ca-aluminates and/or Ca-silicates, such as those described in WO 2000/021489, WO 2001/076534, WO 2001/076535, WO 2004/037215, WO 2004/00239 and WO 2003/041662 and U.S. Pat. No. 6,620,232, U.S. Pat. No. 6,969,424, U.S. Pat. No. 7,025,824. and U.S. Pat. No. 7,048,792. Said materials have been proposed for use in dental applications as well as in orthopedic applications. The most frequently used chemically bonded ceramics are those based on Ca-phosphates, and blood clotting at the surface of these materials is generally been interpreted as a good sign for possible bone formation at the implant surface. A general description of ceramics used as biomaterials can be found in “An Introduction to Bioceramics,” by L. Hench and J Wilson, World Scientific Publ. Co., Singapore, Hong-Kong, the contents of which in relevant parts is incorporated herein by reference.

The process that leads to thrombosis formation as blood contacts an artificial surface depends on a range of factors coupled to the material and its surface characteristics, the rheology and the biological aspects, commencing with the initial step of protein adsorption. The most widely used chemical compounds to reduce thrombogenecity and increase the haemocompatibility of stents are those based on phosphorylcholine (PC) or heparin substances. Heparin and PC can be coated upon the desired implant, but need some linking to the implant material. Heparin is often placed on top of another organic coating, e.g. polyamine (PAV). PC, on the other hand, is bound to the implant via linking of e.g. 3-amino-propyltrimethoxysilane (3-APTMS). Heparin based materials can also be delivered as a drug by systemic administration.

Heparin and PC also reduce the adhesion of bacteria. This is due to the chemistry of the compounds, which substantially reduces the tendency of protein adsorption.

A summary of the status for PC and Heparin-based materials is presented in a recent PhD thesis entitled “Characterization of surfaces designed for bio-medical applications,” by Emma Kristensen, Uppsala University, ISBN 91-554-6546-3, p 14-22. (2006).

To substantially reduce or avoid thrombogenecity of the surface of an implant it would be desirable to be able to use materials in direct blood contact not having any clotting tendency, thereby substantially reducing or avoiding the use of drugs, such as phosphorylcholine and heparin substances. Accordingly, it is an object of the present invention to improve the haemocompatibility of a blood tissue implant to such an extent as to substantially reduce or avoid the need of undesired medical treatment by medication. It is also an object to avoid the need of an additional linking substance.

### SUMMARY OF THE INVENTION

It has surprisingly been found that a coating layer as such, based on calcium aluminate compounds as a biomaterial, also exhibits the desired haemocompatibility and markedly reduced thrombogenecity. The coating can be applied directly to the substrate, without any additional linking substances as required in the prior art. Also, the inventive coating does not require the use of any drugs and/or medication, as in the prior art. The coating material works as an improved surface of the blood tissue implant and at the same time as a preventing surface against blood clotting.

An additional feature of the invention is a good biocompatibility of the coating also towards soft tissue. Accordingly, a stent exposed to both blood and soft tissue in the human or animal body, exhibiting a coating of the invention will therefore have a good biocompatibility also to the soft tissue.

The resulting coating of hydrated Ca aluminate is stable, and loss of the coating material over time of use is virtually zero, in contrast to the conventionally used PC and heparin based coating materials.

Also, the hardness and strength of the inventive coating are considerably higher than for the prior art PC and heparin based coatings.

Moreover, the inventive coating can be applied by means of CVD and PVD, thus allowing for application of very thin layers, such as of about 1 to about 20 μm, and also for carefully controlling the thickness thereof.

Accordingly, in one aspect the present invention relates to the use of a powdered composition based on calcium aluminate and/or solid solution thereof forming a chemically bonded ceramic biomaterial on hydration, for forming a coating on metals, organic polymers and/or ceramics, to avoid or reduce thrombogenecity and/or adsorption of bacteria.

In another aspect the present invention relates to an implant device to be in contact with the blood flow of a mammal, which device has a reduced thrombogenecity and adsorption of bacteria. Such devices are set forth in claims 11-21.

In yet an aspect the present invention relates to a method for preparing a device to be in contact with the blood flow of a mammal, wherein said device exhibits a reduced thrombogenecity and adsorption of bacteria. Such method is set out in claims 5-9.

In yet an aspect the present invention relates to a method of implantation of a device to be in contact with the blood flow of a mammal exhibiting reduced thrombogenecity and adsorption of bacteria. Such method is set out in claim 10.

Further aspects, advantages and details of the invention are described in the detailed description, Examples and in the claims.

### DETAILED DESCRIPTION OF THE INVENTION AND PREFERRED EMBODIMENTS

For the purpose of the present disclosure the term “blood tissue implant” is intended to refer to an implant which is exposed to blood when implanted into the human or animal body, and more particularly a blood vessel implant. Blood in turn is considered a tissue of the human or animal body. Examples of blood tissue implant are stents, heart implants, e.g. heart valves, transfer implants into blood vessels, such as for dialysis machines, needles and long-term needles, and also grafts.

The surface of the implant to be coated may be made of metals, organic polymers and/or ceramics.

For compounds of the CaO—Al₂O₃—H₂O (CAH) system, it has surprisingly been found that the hydrated surfaces of a coating thereof react in a similar way found in PC and heparin based technology to avoid blood clotting. While not wishing to be bound to any specific theory it is believed that this is due to the partial dual charge characteristics of the hydrates formed in the CAH coatings, the wettability and the increased amount of water molecules just outside the implant surface, and that Ca-aluminate as a hydrate thus has a similar zwitter-ion mechanism as PC, which hinders the start of the cascade of coagulation. Therefore, water molecules will attach strongly to the surface due to the partial positive and negative charge, and proteins cannot reach the surface for anchoring. This contributes to reduced protein adsorption and possibly also to reduced adhesion of bacteria. The CAH system provides coatings with favorable general features with regard to adhesion to the substrate (high shear strength) and wear resistance. Favorable phases in the hydration process are katoite and gibbsite. These phases are formed at hydration processes above approximately 30° C.

General aspects of suitable Ca-Aluminate systems for use in the present invention are known in the prior art and have been described in e.g. U.S. Pat. No. 7,048,792 and U.S. Pat. No. 7,074,223.

The techniques used to apply the coating depend on the geometry of the device to be coated. For devices with relatively simple geometry CVD and PVD techniques can be used. For more complex geometries or extended devices sol-gel processing and precipitation techniques are preferred.

The coating can be applied to the implant device, such as a stent, or heart valve, in the form of a powder, or a solution/slurry thereof, into which the device conveniently can be immersed, as will be explained in more detail in the Examples.

In one embodiment the applied coating is completely hydrated before in vivo use, so that the desired katoite and gibbsite phases are formed before implantation.

In another embodiment of the invention the applied coating is only partially hydrated before in vivo use. This will favor the contact of the coating with the soft tissue, since the coating material will be more active and thus interaction with the tissue will occur. In this case a pH reducing agent may advantageously be added, since otherwise using partially unhydrated Ca-aluminate the pH may be undesirably high. The unhydrated Ca-aluminate phase immediately starts to hydrate and these hydrates show as the prehydrated Ca-aluminate surface increased haemocompatibility.

In a preferred embodiment the Ca-aluminate composition is (CaO)ₐ (Al₂O₃)_{b}, wherein the ratio a:b is in the interval from 0.5:1 to 3:1.

In a preferred embodiment the Ca-aluminate composition is 3CaO Al₂O₃ (C3A).

### EXAMPLE 1

Ca-aluminate (C3A) was synthesized at 1400° C. in a conventional sintering furnace for 6 h. The material was crushed and thereafter milled for 72 h in a rotating mill using ceramics containers and with the milling media of Silicon nitride balls (15 mm in diameter) and iso-propanol as liquid. After thin film evaporation the powder was completely dried, the powder was poured into distilled water, to obtain a supersaturated solution. Implant devices of commercially pure titanium were dipped into the solution for different time periods. The solution was kept at 37° C. The layer thickness was qualitatively found to depend on the time of immersion, and was <10 μm after 24 hrs. This layer was used in the evaluation in Example 3 below.

### EXAMPLE 2

In complementary studies the powder based on CaO Al₂O₃ (CA)—synthesized at 1375° C.—was pressed to pellets and used as target in a PVD process, with the following features:

Size of the target: 10×10 cm

Density of the target: 60% of theoretical density

PVD instrument: RF magnetron

Using the PVD process the Ca-aluminate target material was transferred to an implant device surface, in this case the surface of a thin Ti-plate, commercially pure titanium (1×4 mm). The thickness of the Ca-aluminate coating thus obtained was approximately 0.3 micrometer.

The coated surface was heat treated (i.e. hydrated) in water at 37° C. and at 60° C. X-ray diffraction of the coating revealed the presence of katoite, 3CaO Al₂O₃ 6H₂O (C3AH6), and gibbsite, Al(OH)₃, as the only detectable phases. At temperatures below 30° C. the unstable 8-phase, CaO 2Al₂O₃ 8H₂O (CA2H8), may be developed.

### EXAMPLE 3

The coated devices obtained in Examples 1 and 2 were tested in a model system with human whole blood using a close circuit Chandler loop model. The model exposes the test material to fresh human whole blood. Reference material: Immobilized functional heparin. The loops are rotated at 33 rpm in a 37° C. water bath for 60 minutes. After the incubation, the blood and the coating surfaces were investigated with special attention to clotting reactions. Blood samples were collected and supplemented with EDTA for cell count analysis. Blood from the loops was centrifuged to generate plasma for analysis of thrombin-antithrombin complex (TAT), C3a and TCC (Terminal complex of complement) complement marker. No or only limited clotting behaviour of the coatings were observed. Three test runs were performed with different blood donors. At each occasion, the material was tested in two loops, including a control loop without test material (control), making a total of 9 loops per test run. For each loop, a blood sample was immediately analysed in a cell counter. A plasma sample was prepared by centrifugation and immediately frozen to −70° C. A range of tests was performed on the blood/plasma. Also the donor blood was tested, without having passed the loop test (baseline). The collected blood samples and the coated surfaces were first evaluated with respect to clotting by the eye. The blood was positioned on a cloth to detect possible clots or contaminations from the test materials The clotting behaviour and platelet count of the Ca-aluminate material was comparable to both control and baseline, showing very low clotting and maintaining high platelet count numbers, around 200 for the Ca aluminate coating and the references. The concentration of thrombin-antithrombin complex (TAT) was largely unaffected for the test procedure for the Ca-aluminate material. The Ca-aluminate material should not be expected to cause thrombotic complications in view of its very low tendency to induce activation of the coagulation system.

1. A method for reducing or avoiding thrombogenecity of a surface of a metal, an organic polymer or a ceramic and/or reducing or avoiding adsorption of bacteria to said surface comprising applying to said surface a coating of a powdered composition comprising calcium aluminate and/or a solid solution of calcium aluminate which forms a chemically bonded ceramic biomaterial by hydration thereof.

2. The method of claim 1, wherein the coating is formed on a blood tissue implant.

3. The method of claim 1, wherein the calcium aluminate has a composition of (CaO)ₐ (Al₂O₃)_{b}, wherein a ratio of a/b is 0.5 to 3.

4. The method of claim 1, wherein the calcium aluminate has a composition of 3CaO Al₂O₃.

5. A method of preparing a coating on a blood tissue implant having reduced thrombogenecity and reduced adsorption of bacteria comprising the steps of:
a) providing a blood tissue implant;
b) applying a coating of a powdered composition comprising calcium aluminate and/or a solid solution thereof forming a chemically bonded ceramic biomaterial by hydration thereof to a surface of the implant; and
c) at least partially hydrating the coating formed in b) so as to form phases of katoite and/or gibbsite.

6. The method of claim 5, wherein in step b) a solution or slurry of the powdered composition is applied to a surface of the implant.

7. The method of claim 5, wherein in step b) the powder coating is applied using PVD, CVD or sol-gel processing.

8. The method of claim 5, wherein in step c) the coating is hydrated in a water-containing solution and/or in highly humid air having a RH>60% at a temperature above 30° C.

9. The method of claim 5, wherein the coating in step b) is precipitated from a calcium aluminate water solution at a temperature above 30° C.

10. A method of implanting a blood tissue implant provided with a coating having reduced thrombogenecity and reduced adsorption of bacteria comprising the steps of:
a) providing a blood tissue implant;
b) applying a coating of a powdered composition comprising calcium aluminate and/or a solid solution thereof forming a chemically bonded ceramic biomaterial by hydration thereof to a surface of the implant;
c) at least partially hydrating the coating formed in b) so as to form phases of katoite and/or gibbsite; and
d) implanting the coated implant into a mammal in need thereof.

11. A blood tissue implant comprising a coating comprising calcium aluminate and/or a solid solution of calcium aluminate forming a chemically bonded ceramic biomaterial by hydration thereof, obtained by the method of claim 5, wherein the coating after hydration has a thickness of less than 20 μm.

12. The blood tissue implant of claim 11, wherein the coating after hydration has a thickness less than 5 micrometers.

13. The blood tissue implant of claim 11, wherein the coating after hydration has a thickness less than 1 micrometer.

14. The blood tissue implant of claim 11, wherein the coating contains the phases of katoite and/or gibbsite.

15. The blood tissue implant of claim 11, wherein the coating containing the hydrated phases has crystal sizes less than 100 nm.

16. The blood tissue implant of claim 11, wherein the coating is partially hydrated to a degree above 50%.

17. The blood tissue implant of claim 11, wherein the coating further comprises a pH reducing agent.

18. The blood tissue implant of claim 11, further comprising a complementary drug.

19. The blood tissue implant of claim 18, wherein the complementary drug is a complementary thrombogenecity reducing drug.

20. The blood tissue implant of claim 11, selected from the group consisting of a graft, a stent, a heart implant and a transfer implant into a blood vessel.

21. The method of claim 5, wherein the mammal is a human.

22. The method of claim 10, wherein the mammal is a human.
